(19) United States
(12) Patent Application Publication
(10) Pub. No.: US 2017/0327865 A1
(43) Pub. Date: Nov. 16, 2017

(54) Personalized Glucose and Insulin Monitoring System

(71) Applicant: eNano Health Limited, Hong Kong SAR (HK)

(72) Inventors: Patrick Shau-park Leung, Arcadia, CA (US); Ho In Chow, Hong Kong (HK); Christine Yau, Hong Kong (HK)

(73) Assignee: eNano Health Limited, Hong Kong SAR (HK)

(21) Appl. No.: 15/666,699

(22) Filed: Aug. 2, 2017

Related U.S. Application Data
(63) Continuation-in-part of application No. 15/469,138, filed on Mar. 24, 2017.
Application No. 15/469,138 is a continuation-in-part of application No. 15/056,163, filed on Feb. 29, 2016.

(51) Int. Cl. C12Q 1/54; G01N 33/487; G01N 33/53; C12Q 1/68; G01N 33/66; G01N 33/74

(52) U.S. Cl. CPC C12Q 1/54; G01N 33/66; G01N 33/48792; G01N 33/74; C12Q 1/6876; G01N 33/5308; G01N 33/5306; G01N 2333/62; G01N 2400/00

(57) ABSTRACT

The claimed invention provides personalized glucose and insulin information to a user in need thereof. Non-invasive body fluid capture techniques utilize saliva to provide body levels of glucose and insulin as well as optional pharmaceutical ingestion coordinated over time. Saliva captured on cellulose strips are analyzed in real time using oxidation and aptamer conjugate hybridization together with traditional analytical chemistry techniques including liquid chromatography/mass spectrometry (LC/MS) and coordinated against time of pharmaceutical administration. By embracing the P4 (Participatory, Personalized, Predictive, and Preventive) health management method the patient can determine glucose and insulin related wellness levels and if a pharmaceutical is having the correct and desired effect for maximum therapeutic benefit.

### TECHNICAL FIELD

The claimed invention relates to biomedical healthcare patient monitoring based upon the P4 (Participatory, Personalized, Predictive, and Preventive) health management method. With greater particularity, the claimed invention addresses personalized monitoring of diabetic, pre-diabetic and general health and wellness based on enhanced glucose plus insulin monitoring in the presence and absence of prescription pharmaceuticals. Enhanced patient alerting and artificial intelligence data interpretation is also incorporated in enhanced illustrative embodiments.

### BACKGROUND ART

Traditional biomedical monitoring of patient glucose levels often utilizes consumer ‘finger prick’ test strips which measure glucose present in the user's blood. Corresponding glucose levels obtained by visually measured color change can be imprecise and infrequent due pain and discomfort associated with blood sample collection. Measuring insulin, however, is often clinical in nature with results ordered by a doctor in a hospital or medical office setting and performed in a centralized laboratory setting. Even when patients are informed as to their insulin blood levels it is often through the lens of the primary medical provider.

Using traditional methods, if a patient wishes to know detailed information about insulin levels in the body they must first schedule an office visit. Absent an emergency, such visits usually take place weeks to months after the request is made. To determine body levels of insulin, blood is drawn and sent to an outside laboratory. Several days later the results are reported back to the primary healthcare physician who interprets the laboratory results and provides a high level summary to the patient.

Despite the rapid expansion of ‘big data’ healthcare information, patients are rarely the owners or curators of their own healthcare information leading to reduced choices and far fewer options in healthcare data portability for independent analysis at home or when seeking out alternate providers.

### SUMMARY OF INVENTION

### Technical Problem

Current systems for monitoring of both glucose and insulin levels in the human body are centralized and exclusionary and not simultaneous using current methods. They are not participatory apart from the blood sample that the patient provides for testing. Reporting insulin and glucose levels are presently not personalized in that apart from the unique data itself released by a medical healthcare provider, the medical service provider controls the manner, method and timing of body insulin information release.

Drawing of patient blood in a clinical setting creates a number of challenges including sample perishability, hazardous waste disposal and personal bias against invasive procedures. Solution to Problem

By embracing the P4 (Participatory, Personalized, Predictive, and Preventive) health management method, the claimed invention provides patient engaging glucose plus insulin level data. In a preferred illustrative embodiment, pharmaceutical administration information is additionally provided. By utilizing patient saliva samples which are collected and transported to a centralized analysis facility, glucose and insulin levels are accurately captured and rapidly delivered to the patient using a smartphone, smartwatch or personal computing device.

A diabetic, pre-diabetic or wellness interested individual's insulin and glucose level information is non-invasively obtained by saliva samples collected on disposable sample means including paper strips. Using traditional analytical laboratory equipment including Liquid Chromatography/Mass Spectrometry (LC/MS) and Enzyme-linked immunosorbent assay (ELISA), measured wellness indicator levels are obtained and reported back to the patient directly using secure internet data transmission techniques.

### Advantageous Effects of Invention

By empowering the patient to become curators their own wellness information including glucose and insulin levels using saliva, predictive and preventative wellness is enabled. The claimed invention is distinguishable from using traditional blood level monitoring due to the powerful wellness knowledge enabled by simultaneous glucose and insulin level identification and lack of body ‘finger prick’ invasive blood collection. In particular enhanced embodiments, by calculating time of pharmaceutical administration against the results obtained by the saliva/paper sample conjugate analyzed by ELISA and LC/MS, additional wellness is obtained through monitoring and administration of proper pharmaceutical administration.

By utilizing disposable cellulose strips to detect saliva, medical waste is greatly reduced. As an additional intended benefit, patient privacy is maintained as no patient identifiable information needs to be included in the test strip when it is independently analyzed.

### BRIEF DESCRIPTION OF DRAWINGS

The accompanying drawings are included to better illustrate exemplary embodiments of the claimed invention.

FIG. 1 is a top level schematic illustration of saliva derived glucose and insulin level test strip with enhanced functionality.

FIG. 2 is a side view schematic illustration of saliva derived glucose and insulin level test strip with additional functionality.

FIG. 3 is a graphical chart illustration of patient glucose and insulin levels over time.

FIG. 4 is a flowchart illustrating a preferred embodiment of the claimed invention.

FIG. 5 is a graphical chart illustration of saliva derived glucose and insulin levels with body pharmaceutical content plotted by time.

FIG. 6 is a flowchart illustrating a preferred embodiment of the claimed invention.

FIG. 7 is a flowchart illustrating a preferred embodiment of the claimed invention.

### DESCRIPTION OF EMBODIMENTS

P4 Medicine is Predictive, Preventive, Personalized and Participatory. Its two major objectives are to quantify wellness and demystify disease. In the illustrative examples contained herein, the aims of P4 Medicine are achieved by combining end-user analysis of saliva based glucose and insulin health metrics together with follow-on lab analytics of the same saliva sample to determine body levels of glucose and insulin and other indicators such as DNA, miRNA, body chemistry indicators and administered pharmaceuticals. In a first illustrative example, user health data is gathered by smartphone to determine body glucose and insulin levels and related health details such as time of food intake.

In the illustrative embodiments the glucose and insulin measuring test strips report glucose and insulin levels to the end-user for personalized and participatory wellness monitoring. The saliva capture cellulose test strip is a multi-part and multi-layer disposable indication device utilizing lateral flow to isolate and expose multiple body wellness and diagnostic regions. While the illustrative examples depict a user data capture upper layer and remote analytical measurement lower layer, the specific orientation of the respective layers are by design preference only rather than by limitation and can be reoriented as desired.

The same test strip subsequently analyzed using standard analytical equipment, however, provides the opportunity for predictive and preventative health screening based upon detection of pharmaceuticals and their carriers as well as DNA, RNA and protein indicators of body health as well as the presence or absence of harmful bacteria, viruses and other disease carriers in addition body wellness indicators such as insulin.

### EXAMPLES

### Example 1

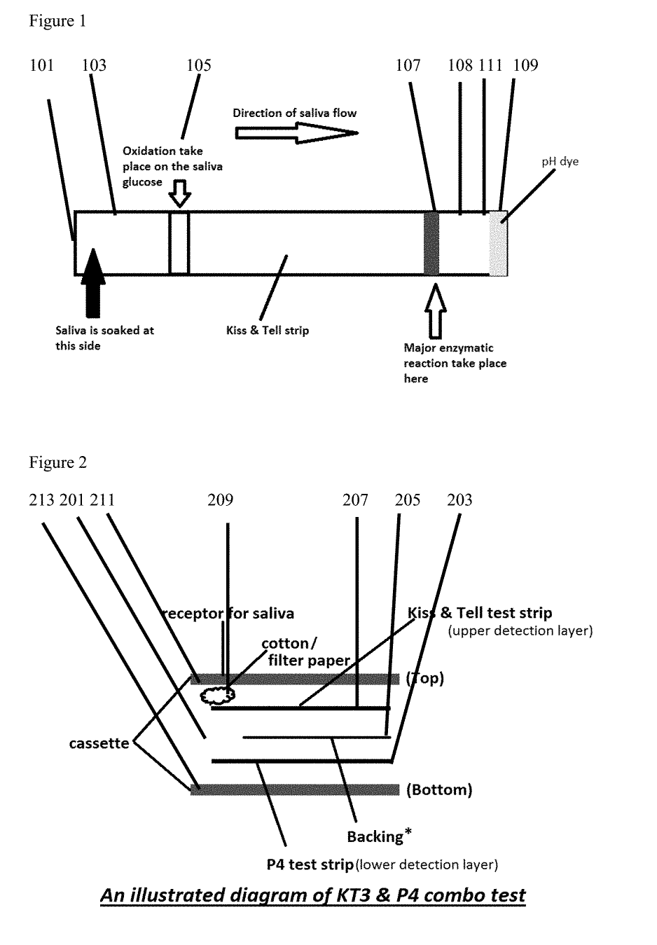

The claimed P4 wellness platform is based upon salivary capture and analysis using one or more disposable cellulose/paper test strips. FIG. 1 depicts the cellulose test strip in the most simple form, where salivary test strip (101) captures saliva (not shown) at salivary capture area (103) which is distributed by lateral flow into oxidation region (105) and onto enzymatic region (107) concluding with pH region (109). In the first illustrative embodiment the enzymatic analysis provides measurable salivary glucose levels and may additionally incorporate insulin antibody indicator region (108) as well as optional aptamer indicator region (111).

In the first illustrative example, body glucose levels are captured by placing test strip (101) in a user's mouth (not shown) for two minutes to distribute saliva (not shown) to test strip (101). Adequate saliva capture is confirmed by illumination of pH region (109). In the first illustrative example, the user waits an additional three minutes upon which a measurable color change takes place at enzymatic region (107). Salivary glucose levels may be estimated by user color comparison visually or by computer analysis by a smartphone type device (not shown).

In the first illustrative embodiment, the salivary test strip may be single purpose as illustrated by salivary test strip (101) depicted by FIG. 1 or multi-purpose as illustrated by multi-function salivary test strip (201) depicted in FIG. 2. FIG. 2 multi-function salivary test strip (201) is multi-layer with top analytical layer (207), layer divider (205) backing and lower analytical layer (203). Saliva access is provided through optional cassette housing (213) with salivary receptacle (211) which distributes saliva (not shown) through optional saliva wicking material (209) which can be cotton, filter paper or other material suitable for distribution of saliva.

Both glucose and insulin levels are derived from the user's saliva. Saliva is unlike blood in that it is stable at room temperature for long periods of time, safe to collect and transport and not considered medical waste. It is a direct and intended consequence of the claimed invention that measuring glucose and insulin from saliva provides more immediate and relevant composite wellness picture owing to the delay between meal ingestion and corresponding blood levels. In the first illustrative example the user is able to capture glucose information shortly after sample exposure. To obtain body insulin levels sample test strip (201) is mailed to a remote location for further analysis using traditional laboratory equipment such as LC/MS and spectrophotometer for ELISA screening. In an additional embodiment, insulin levels are additionally derived from antibodies corresponding to insulin added to the upper level in addition to glucose enzymatic reaction area. As a result, ‘on the spot’ results can be better calibrated and confirmed by independent glucose test measurements. Insulin levels are also derived with both results reported back to the user via smartphone data transmission.

### Example 2

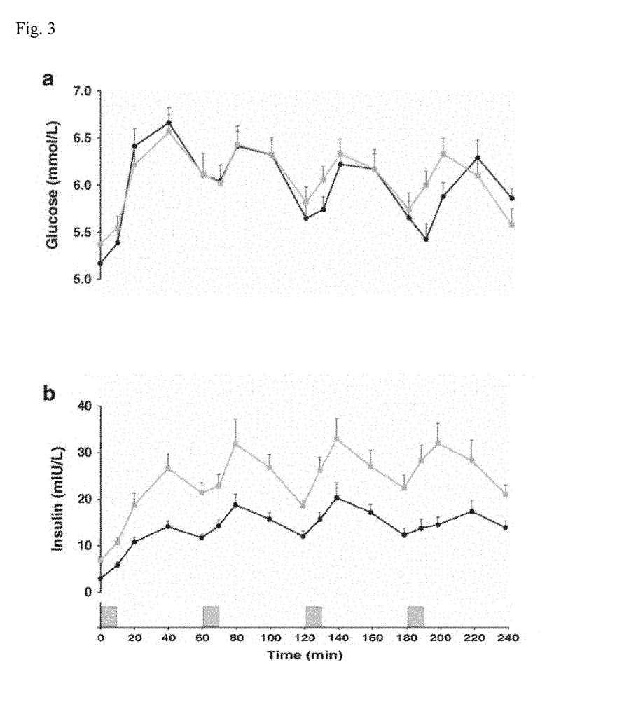

An ultimate objective of the claimed invention as further detailed in illustrative embodiment Example 2 is the user's access to composite insulin and glucose data over time and coordinated with the user's meal times. Traditional glucose ‘finger prick’ disposable strips do not meaningfully inform the user as to underlying insulin levels. FIG. 3 illustrates glucose coordination with insulin level divergence between normal and obese individuals represented with round icons for normal and square icons for obese. The graphs showing the means±s.e.m. (standard error of the mean) of plasma glucose (a), insulin (b) concentrations for normal-weight (circle) and obese subjects (square) following four 706 kJ 10-min meals (time 0, 60, 120 and 180 minutes). These two graphs are showing the changes of glucose and insulin in normal-weight and obese subjects after taking the same meals at a regular period identified by shaded bars on the x-axis. The graphical illustration clearly shows that the glucose concentration of normal-weight and obese subjects are similar after taking the same meal. However, the insulin concentration of obese subject increases to a greater extent than the normal-weight subject.

In preferred embodiment Example 2 detailed below a user captures glucose data in near real time utilizing a plurality of multi-function salivary test strips which are subsequently remotely analyzed for insulin and optionally with additional glucose reporting functionality. By using a smartphone to input time of saliva sample data capture as well as most recent mealtime, glucose results are immediately captured and insulin results are returned to generate results allowing the user to determine general wellness indicators including healthy and divergent pre-diabetic insulin levels placing the user at risk from obesity.

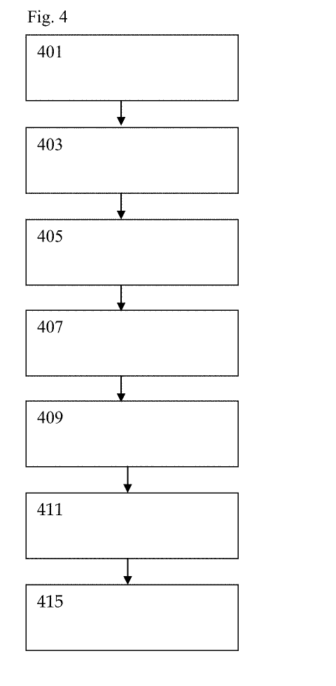

FIG. 4 illustrates the process of utilizing the claimed invention to manage wellness by deriving glucose and insulin levels coordinated by meal times. Sample preparation step (401) begins with the user placing saliva on a sample collection means and the system stores the time of saliva sample capture. In the illustrative embodiment the saliva sample is captured by the user on a cellulose strip which may be enhanced with additional wellness indicators as previously detailed. Glucose data capture step (403) is achieved by a user taking a smartphone picture of a saliva exposed sample strip and inputting the most recent meal time and optionally meal consumption information. In the illustrative example a smartphone is used which may also be a personal computer or dedicated device. During glucose data capture step the test strip is associated with a user's unique and anonymous strip data identifier which may be a two-dimensional or QR bar code on sample strip packaging or determined by alternate forms such as machine vision or NFC communication. The saliva sample cellulose strip is sent by mail or otherwise transported to a central analysis facility and analyzed by liquid chromatography and mass spectrometry (LC/MS) during sample chemical analysis step (405) to determine body levels of insulin as well as optional ELISA analysis for glucose. While the illustrative example utilizes a centralized LC/MS analysis platform other foreseen and intended variants may utilize localized dedicated analysis platforms.

The remainder of Example 2 illustrated by FIG. 4 takes place in a computational or cloud computing environment. During data analysis step (407) body levels of insulin and glucose are analyzed against time of sample preparation and meal consumption together with other optional wellness indicators. Data transmission step (409) transmits the user body insulin level results to the user's preferred computational device including smartphone and smart watch. Data reporting step (411) provides the user with body insulin and glucose as a function of time. Optional data alert/feedback gathering step (413) reports abnormal or medically dangerous wellness indicator levels to the user as well as medical providers and designated family members and provides an opportunity for gathering user feedback. Data mining step (415) provides a deeper analysis into body insulin production as a function of time and behavior as greater data is collected by the system.

In a more specific illustrative embodiment, sample preparation step (401) begins with a user placing a saliva sample collection means in the mouth to collect saliva and takes a digital photo of the paper strip with a smartphone. Saliva is exposed to an oxidation region on the strip followed by an enzymatic color change. After exposure to saliva the user takes a photo of the strip which captures the time of strip exposure and provides time and body glucose data to the system. The saliva capture means is associated to the system by way of 2D or QR bar code, machine readable numbers or other identifiable characteristics. Glucose data capture step (403) takes place with the user inputting additional personal details including height, weight and age along with meal consumption details including time of meal. Input may be through smartphone, smart watch, stand alone computer or other dedicated computing device. After saliva exposure and smartphone photo capture the sample is placed into a prepaid envelope provided during purchase in the consumer packaging and is sent by mail or otherwise transported to a central analysis facility and analyzed by liquid chromatography and mass spectrometry (LC/MS) during sample chemical analysis step (405). Unlike blood or other biological material collection, the sample is safe at room temperature and does not create hazardous waste handling concerns.

Use of the claimed system is an iterative process, the more times the user provides results the more powerful the data becomes for user lifestyle wellness management. Optional data alert/feedback gathering step (413) is available to alert the user to deviations from normal insulin level generation. Feedback can also be obtained as a result of change in behavior which can be observed and reported back to the user. Data mining step (415) provides a deeper analysis into insulin level measurement as a function of time and behavior as greater data is collected by the system. While artificial intelligence cloud computing provides a computationally powerful tool, the smartphone/smart watch user interface report of data aggregation is intended to be simple by design.

### Example 3

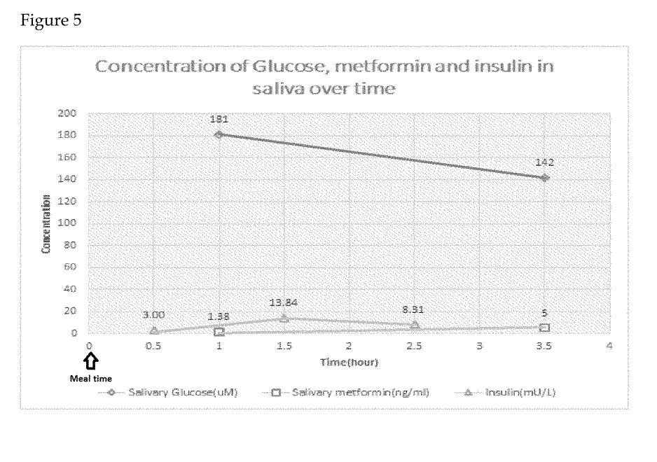

Enhanced functionality of the disposable saliva capture strip platform is demonstrated in FIG. 5. Example 3 utilizes the salivary test strip analysis detailed in previous examples and enhances the functionality with subsequent LC/MS analysis of the saliva sample for pharmaceutical presence or absence. Representative data for Example 3 is provided in Table 1 below and graphically illustrated in FIG. 5.

TABLE 1

| | Salivary | Salivary | |
|---|---|---|---|
| Time(hour) | Glucose(uM) | metformin(ng/ml) | Insulin(mU/L) |
| 0.5 | | | 3.00 |
| 1 | 181 | 1.38 | |
| 1.5 | | | 13.84 |
| 2 | | | |
| 2.5 | | | 8.31 |
| 3 | | | |
| 3.5 | 142 | 5 | |

Where time = 0 is meal time.

FIG. 5 depicts the real-time determination of salivary glucose plotted against meal time together with saliva derived insulin levels as well as body levels of the pharmaceutical metformin. Both insulin and metformin levels are determined remotely by traditional lab equipment including LC/MS and reported back to the user by smartphone. The disposable cellulose saliva sample allows for determination of all three wellness indicators from the same test strip.

### Example 4

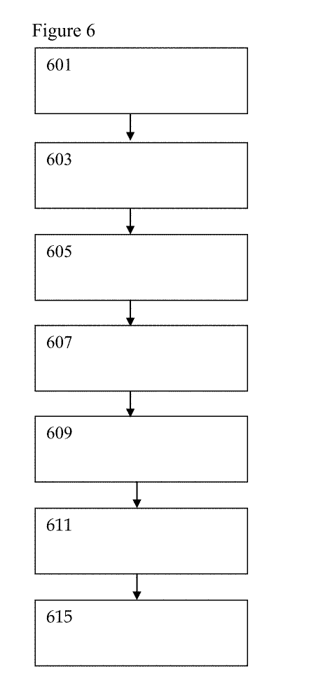

In a further illustrative example as detailed in FIG. 6, expanded personalized wellness information is obtained by substitution of an indicator aptamer in place of enzymatic glucose sensing. In addition, LC/MS pharmaceutical detection is supplemented with DNA and RNA sequencing of the saliva sample. Sample preparation step (601) begins with a user in need of medical monitoring placing a saliva sample collection means in the mouth to collect saliva and takes a digital photo of the paper strip with a smartphone. The strip contains one or more aptamers embedded in the saliva collection device which undergoes an optical or machine readable detection upon hybridization. After exposure to saliva the user takes a photo of the strip which captures the time of strip exposure and provides capture time and aptamer data to the system. The saliva capture means is associated to the system by way of 2D bar code, machine readable numbers or other identifiable characteristics. Pharmaceutical data capture step (603) takes place with the user inputting pharmaceutical details of dosage and latest time of administration. Input may be through smartphone, smart watch, stand alone computer or other dedicated computing device. After saliva exposure and smartphone photo capture the sample is placed into a prepaid envelope provided during purchase in the consumer packaging and is sent by mail or otherwise transported to a central analysis facility and analyzed by liquid chromatography and mass spectrometry (LC/MS) as well as genetic sequencing during sample chemical and genetic analysis step (605). Unlike blood or other biological material collection, the saliva sample is safe at room temperature and does not create hazardous waste handling concerns.

Data analysis step (607) takes place in a cloud computing environment to analyze body levels of pharmaceuticals against time of administration and aptamer indicated and genetic sequencing indicated conditions to determine the best time when the drug should be taken for optimal beneficial effect. In a foreseeable and intended embodiment the presence or absence of pharmaceutical carriers as well as multi-drug detection is carried out by the LC/MS system to determine if the pharmaceutical product is counterfeit and if the user is at risk from multi-drug cross reactions. In an intended alternate embodiment the presence or absence of illicit substances is also detected. Furthermore, the genetic sequencing and data analysis of the saliva sample allows for detection of bacterial and viral infections by screening for miRNA and DNA targets of interest.

The results are wirelessly transmitted over the internet during data transmission step (609) and the user's smartphone or smartwatch user interface displays a high level metadata analysis during data reporting step (611). Unlike traditional Physician's Desk Reference (PDR) or pharmaceutical packet insert materials, the data is presented in plain language and can be as simple as “You've been taking your medication regularly but it looks like you may have a bacterial infection as well. Have you noticed a health change or consulted your physician?”

Use of the claimed system is an iterative process, the more times the user provides results the more powerful the data becomes for user lifestyle wellness management. Optional data alert/feedback gathering step (613) is available to alert the user, designated family members and medical providers if critical overdose, dose omission or counterfeit pharmaceutical product is detected during sample analysis by screening for both pharmaceutical product as well as commonly used pharmaceutical carriers. Feedback can also be obtained as a result of change in behavior and can be as simple as the system asking the user “It looks like you inadvertently took a double dose of your product last week. Now that you are taking your pill at its recommended levels again, do you feel better?” Data mining step (615) provides a deeper analysis into drug administration as a function of time and behavior as greater data is collected by the system. While artificial intelligence cloud computing provides a computationally powerful tool, the smartphone/smart watch user interface report of data aggregation is intended to be simple by design. Aggregate results in this illustrative example are provided in a simple format for improved user personalized health.

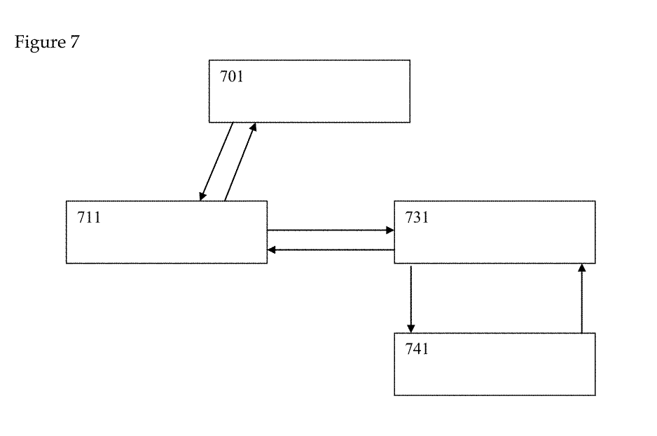

The system of the second example is illustrated in FIG. 7. Results from saliva sample collection device (741) are captured by personal communication device (731) incorporating one or more central processing units, one or more cameras and internet connection means. Health sample analysis hardware (701) further analyzes saliva sample collection device (741) with results communicated through health sample interpretation software, artificial intelligence element and cloud computing element (711) for interpretation and communication of saliva sample health care results (not shown). Saliva sample collection device (741) may optionally contain one or more health sample detection chemicals as well as one or more health sample detection aptamers. Sample analysis hardware includes chromatography and mass spectrometry functionality and can additionally include genetic sequencing functionality.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

### INDUSTRIAL APPLICABILITY

The claimed invention has industrial applicability in the biomedical arts. In particular, the claimed invention is directly relevant to individual monitoring of glucose and insulin for personal wellness.

### CITATION LIST

### Patent Literature

This patent application is a continuation-in-part and claims priority to U.S. patent application Ser. No. 15/469,138 filed Mar. 24, 2017 to Patrick Shau-park Leung entitled “Public personalized mobile health sensing system, method and device” which is a continuation of U.S. patent application Ser. No. 15/056,163 filed Feb. 29, 2016 to Patrick Shau-park Leung entitled “Mobile automated health sensing system, method and device”.

### Non-Patent Literature

Illustrative discussions concerning the obese versus healthy generation of insulin are further detailed by Seyssel K, Allirot X, Nazare J-A, et al (2015) Plasma acyl-ghrelin increases after meal initiation: a new insight. European Journal of Clinical Nutrition 70:790-794. doi: 10.1038/ejcn.2015.181.

We claim:

1. A personal saliva glucose measuring device comprising:
Cellulose substrate, saliva gathering region, saliva glucose oxidation region, enzymatic reaction area and pH dye.

2. The personal saliva glucose measuring device of claim 1 additionally comprising one or more backing layers separating an upper detection layer from one or more lower detection layers.

3. The personal saliva glucose measuring device of claim 2 additionally comprising a machine readable unique personal saliva glucose measuring device identifier.

4. The personal saliva glucose measuring device of claim 2 additionally comprising an insulin antibody hybridization region.

5. The personal saliva glucose measuring device of claim 2 additionally comprising an aptamer hybridization region.

6. A personal glucose and insulin monitoring system comprising:
One or more personal saliva glucose measuring devices, a personal communication device incorporating one or more central processing units, one or more cameras, internet connection means, health sample analysis hardware, health sample interpretation software, artificial intelligence element and cloud computing element for interpretation and communication of saliva sample glucose and insulin results.

7. The system of claim 6 wherein said one or more personal saliva glucose measuring device additionally comprises an insulin antibody region.

8. The system of claim 6 wherein said one or more personal saliva glucose measuring device additionally comprises an aptamer hybridization region.

9. The system of claim 7 wherein said health sample analysis hardware additionally comprises chromatography and mass spectrometry functionality.

10. The system of claim 9 wherein said health sample analysis hardware additionally comprises genetic sequencing functionality.

11. A method for personal glucose and insulin data monitoring comprising the steps of:
Sample preparation by exposing a personal saliva glucose measuring device to saliva,
Pharmaceutical data capture by computer device optical acquisition,
Sample chemical analysis,
Sample data analysis step wherein body levels of insulin are analyzed against time of meal consumption,
Data transmission wherein user body glucose and insulin level results are sent to a user's preferred computational device,
Data reporting wherein a user's body glucose and insulin levels are presented as a function of time.

12. The method for personal glucose and insulin data monitoring of claim 11 additionally comprising data alert wherein abnormal insulin levels are reported to the user.

13. The method for personal glucose and insulin data monitoring of claim 11 wherein said computer device is a smartphone.

14. The method for personal glucose and insulin data monitoring of claim 11 wherein said computer device is a smart watch.

15. The method for personal glucose and insulin data monitoring of claim 11 additionally comprising:
Further analyzing said saliva sample during said sample data analysis for pharmaceutical, pharmaceutical carrier and biological wellness indicators utilizing liquid chromatography, mass spectrometry and genetic sequencing techniques.
